(19) United States
(12) Patent Application Publication
(10) Pub. No.: US 2026/0144774 A1
(43) Pub. Date: May 28, 2026

(54) CONTROLLED-RELEASE PHARMACEUTICAL COMPOSITION CONTAINING INDIGO NATURALIS OR ITS MAIN ACTIVE INGREDIENT AND PREPARATION METHOD AND USE THEREOF

(71) Applicant: Centre for Chinese Herbal Medicine Drug Development Limited, New Territories (HK)

(72) Inventors: Zhao Xiang Bian, New Territories (HK); Hor Yue Tan, New Territories (HK); Yiqi Xu, New Territories (HK)

(21) Appl. No.: 19/398,726

(22) Filed: Nov. 24, 2025

(30) Foreign Application Priority Data
Nov. 26, 2024 (CN) 202411702130.X

(51) Int. Cl. A61K 31/404; A61K 9/16; A61K 9/50; A61P 1/04

(52) U.S. Cl. CPC A61K 31/404; A61K 9/1623; A61K 9/1652; A61K 9/501; A61K 9/5026; A61K 9/5089; A61P 1/04

(57) ABSTRACT

The present invention provides a controlled-release pharmaceutical composition containing Indigo Naturalis or its main active ingredient, and a preparation method and use thereof. The controlled-release pharmaceutical composition includes micropellets. The micropellets include an active ingredient and one or more pharmaceutically acceptable excipients, the active ingredient includes Indigo Naturalis, indigo, indirubin, or any combination thereof; wherein the micropellets have an enteric outer layer. The controlled-release pharmaceutical composition provided by the present invention can alleviate the disease condition of inflammatory bowel disease, especially ulcerative colitis, and can reduce or avoid the phagocytosis of the active ingredient by intestinal macrophages, thereby reducing the effect on the patient's liver. The composition exhibits good stability and reduces side effects while maintaining therapeutic efficacy.

### TECHNICAL FIELD

The present invention relates to the field of traditional Chinese medicine and pharmaceutical preparations. Particularly, the present invention relates to a controlled-release pharmaceutical composition containing Indigo Naturalis or its main active ingredient, a preparation method and use thereof.

### BACKGROUND

Inflammatory bowel disease (IBD) mainly includes Crohn's disease and ulcerative colitis. The digestive tract of patients becomes inflamed and ulcerated. Current treatment plans mostly rely on drugs to control the condition, such as using steroidal drugs or immune system suppressants to suppress the immune system, but there are side effects of varying degrees. More severe patients need to undergo surgical treatment. In the past, this disease was more common in Western countries, but in recent years, cases in Asian regions have been increasing. Therefore, there is a need for safer and more effective solutions to alleviate or treat this disease. Indigo Naturalis is a dry powder, lump, or granule prepared from the processing of leaves or stems and leaves of Baphicacanthus cusia (Neeks) Bremek. of the Acanthaceae family, Polygonum tinctorium Ait. of the Polygonaceae family, or Isatis tinctoria of the Brassicaceae family. The main active ingredients of Indigo Naturalis include indigo and indirubin. Modern pharmacological research shows that Indigo Naturalis, indigo, and indirubin have a variety of pharmacological effects. Although Indigo Naturalis and its main active ingredients, indigo and indirubin, have achieved significant efficacy in the clinical treatment of ulcerative colitis, a large number of experimental studies and clinical applications have proven that Indigo Naturalis has liver toxicity, which limits its clinical application. At present, there is no Indigo Naturalis-type formulation that can reduce the side effect on the liver while treating inflammatory bowel disease.

### SUMMARY OF THE INVENTION

In view of the deficiencies of the prior art, the present invention provides a controlled-release pharmaceutical composition for treating inflammatory bowel disease, and a preparation method and use thereof. The controlled-release pharmaceutical composition reduces the adverse effect of active ingredients with liver toxicity on the patient's liver under the condition of intestinal inflammation. The inventors have found through research that indirubin, the main active ingredient of Indigo Naturalis, is the main component related to liver toxicity. Under the condition of intestinal inflammation, macrophage phagocytosis-mediated transport of indirubin leads to liver damage. Therefore, the inventors developed a controlled-release pharmaceutical composition of Indigo Naturalis, indigo, and indirubin that can reduce or prevent macrophage phagocytosis, thereby achieving the therapeutic purpose of maintaining efficacy while reducing side effects for inflammatory bowel disease.

The instant invention provides the following technical solutions.

In accordance with a first aspect of the present invention, there is provided a controlled-release pharmaceutical composition containing Indigo Naturalis or its main active ingredient. The composition comprises micropellets, wherein the micropellets comprise an active ingredient and one or more pharmaceutically acceptable excipients, the active ingredient comprises Indigo Naturalis, indigo, indirubin, or any combination thereof; and the micropellets have an enteric outer layer.

In some embodiments, the micropellets further comprise a blank pellet core, and the active ingredient and the excipient are mixed and coated on the blank pellet core in a solid dispersion manner.

In other embodiments, the micropellets are granules formed by mixing the active ingredient and the excipient, and granulation.

Preferably, the weight ratio of the active ingredient to the excipient is about 1:0.05 to about 1:100, about 1:10 to about 1:50, or about 1:0.1 to about 1:2.

Preferably, the controlled-release pharmaceutical composition comprises 0.5 mg to 500 mg, and more preferably 5 mg to 200 mg of the active ingredient.

In some embodiments, the enteric outer layer is an enteric coating layer coating the micropellets; preferably the enteric coating layer comprises a material selected from hypromellose phthalate (HPMCP), cellulose acetate phthalate (CAP), acrylic resin II, acrylic resin III, and methacrylic acid and methyl methacrylate copolymer, or any combination thereof.

In some embodiments, the weight of the enteric coating layer is 3% to 20% of the weight of the micropellets; more preferably is 8% to 15%.

Preferably, an isolation layer is further coated between the micropellets and the enteric coating layer.

Preferably, the micropellets having the enteric coating layer are further encapsulated in a gastric-soluble capsule.

In other embodimes, the enteric outer layer is an enteric capsule for encapsulating the micropellets.

In accordance with a second aspect of the present invention, there is provided a method for preparing the above controlled-release pharmaceutical composition. The method comprises: a) mixing the active ingredient with the excipient to form a mixture, coating a blank pellet core in a solid dispersion manner to form micropellets, or granulating to form granular micropellets; b) coating the micropellets with an enteric material to form the enteric outer layer on an exterior of the micropellets, resulting in no release or minimal release of drug (less than 20%) in the stomach, and after entering the intestine, the enteric coating begins to dissolve and gradually release the drug under appropriate pH conditions.

In some embodiments, the method further comprises, before step b), forming an isolation layer on a surface of a drug-containing core with an isolation material.

In accordance with a third aspect of the present invention, there is provided a method of treating a disease preferably an inflammatory bowel disease, comprising administering the controlled-release pharmaceutical composition of the present invention to a subject in need thereof.

In some embodiments, the inflammatory bowel disease is ulcerative colitis.

Preferably, the composition is provided in an oral capsule formulation.

Based on the above, the present invention overcomes the shortcomings and deficiencies of the existing technologies, provides an enteric preparation of Indigo Naturalis, indigo, and indirubin with good stability that can reduce or avoid phagocytosis by intestinal macrophages, for the therapeutic use of inflammatory bowel disease. The invention also provides a preparation method for an oral enteric formulation containing Indigo Naturalis, indigo, and indirubin.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIG. 1A is a schematic diagram of a healthy animal model of Example 1, which is used to simulate the liver toxicity of Indigo Naturalis, indigo, and indirubin on healthy subjects;

FIG. 1B is a schematic diagram of relative body weight change of each group of mice in the healthy animal model of Example 1;

FIG. 1C shows H&E staining results of liver sections of each group of mice in the healthy animal model of Example 1, scale bar: 100 μm;

FIG. 1D is a change analysis chart of alanine aminotransferase (ALT) and aspartate aminotransferase (AST) in the serum of each group of mice in the healthy animal model of Example 1;

FIG. 2A is a schematic diagram of a chronic colitis animal model of Example 1, which is used to simulate the effect of Indigo Naturalis, indigo, and indirubin on patients with chronic colitis;

FIG. 2B is a plot of relative body weight change of each group of mice in the chronic colitis animal model of Example 1;

FIG. 2C is a plot of the (C) Kaplan-Meier survival curve of each group of mice in the chronic colitis animal model of Example 1;

FIG. 2D is a change analysis of ALT and AST in the serum of each group of mice in the chronic colitis animal model of Example 1;

FIG. 2E shows the images of H&E staining results of liver sections of each group of mice in the chronic colitis animal model of Example 1, scale bar: 100 μm;

FIG. 3A is a schematic diagram of an animal model of Example 2, clodronate was used to deplete macrophages in the animal body to investigate the effect of indirubin on the liver;

FIG. 3B is a flow cytometry diagram of CD11b+ F4/80+ macrophages in mice receiving liposome control or clodronate of Example 2;

FIG. 3C is a change analysis of ALT and AST in the serum of a control group and a treatment group of Example 2;

FIG. 3D shows the images of H&E staining results of liver sections of the control group and the treatment group of Example 2, scale bar: 100 μm;

FIG. 4A shows the images of raw indirubin and indigo crystals of Example 20; scale bar, 100 μm;

FIG. 4B shows the results obtained from phagocytosis experiment Example 20, which tested whether bone marrow-derived macrophages internalize indirubin and indigo; scale bar: 10 μm;

FIG. 4C shows microscopic images of indirubin enteric-coated micropellets and indigo enteric-coated micropellets prepared according to Example 11; scale bar: 10 μm;

FIG. 4D shows the results obtained from phagocytosis experiment of Example 20, which determined the internalization of indirubin enteric-coated micropellets and indigo enteric-coated micropellets, as shown in FIG. 4C, by bone marrow-derived macrophages; scale bar: 10 μm;

FIG. 5A is a schematic diagram of a chronic colitis animal model of Example 21, 1.8% DSS is used to induce chronic colitis in mice, and each group of mice is given indigo and indirubin micropellets of different particle sizes;

FIG. 5B is a plot of relative body weight change of each group of mice in the chronic colitis animal model of Example 21;

FIG. 5C is a disease activity index (DAI) change curve graph of each group of mice of Example 21;

FIG. 5D is a bar chart showing the resultant colon length of each group of mice of Example 21;

FIG. 5E is a change analysis of ALT and AST in the serum of each group of mice in the chronic colitis animal model of Example 21.

### DETAILED DESCRIPTION OF THE PREFERRED EMBODIMENTS

The following detailed description is provided in conjunction with specific embodiments to facilitate a clearer understanding of the present invention. These embodiments are presented solely for illustrative purposes and are not intended to limit the scope of the invention.

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usual and unavoidable impurities such as side products and components usually resulting from the respective preparation or method for obtaining the material such as traces of further components or solvents. “Consisting of” means that the material solely consists of, i.e. is formed by the respective element. As used herein, the forms “a”, “an”, and “the”, are intended to include the singular and plural forms unless the context clearly indicates otherwise.

The present invention provides a controlled-release pharmaceutical composition containing Indigo Naturalis or its main active ingredients. The composition comprises micropellets, and the micropellets comprise an active ingredient and one or more pharmaceutically acceptable excipients. The active ingredient comprises Indigo Naturalis, indigo, indirubin, or any combination thereof. The micropellets have an enteric outer layer.

The micropellets are spherical entities with a diameter of about 0.5 mm to about 2.5 mm. Preferably, the micropellets are spherical entities having an average diameter of about 1 mm to about 2.5 mm.

In embodiments of the present invention, the active ingredients include Indigo Naturalis, Indigo, and indirubin. In some embodiments, Indigo Naturalis contains at least 1% indigo and at least 0.1% indirubin. Preferably, the controlled-release pharmaceutical composition comprises 0.5 mg to 500 mg, and more preferably 5 to 200 mg of the active ingredient.

The excipient, also known as a formulating agent, can be a conventional pharmaceutically acceptable excipient. The excipient can include, but is not limited to, a filler (a diluent), a wetting agent, a binder, a disintegrant, a lubricant, a coloring agent, etc. In some embodiments, the excipient is selected from sucrose, microcrystalline cellulose, starch, etc., or any combination thereof; the diluent is selected from microcrystalline cellulose, starch, pregelatinized starch, hydroxypropyl methylcellulose, povidone, compressible starch, mannitol, sorbitol, and lactose, or any combination thereof; the disintegrant is selected from starch, low-substituted hydroxypropyl cellulose, carboxymethyl starch, crospovidone, croscarmellose sodium, crospolyvinylpyrrolidone, etc., or any combination thereof; the lubricant is selected from talc, magnesium stearate, calcium dioxide, etc., or any combination thereof; the wetting agent is selected from solvents such as water, ethanol, etc.; the binder or dispersant is selected from syrup, starch paste, sucrose powder, hydroxypropyl methylcellulose, polyvinylpyrrolidone, hydroxypropyl cellulose, xanthan gum, povidone K30-90 series, etc., or any combination thereof; a flow agent such as silicon dioxide or the like.

In the preparation of the micropellets, suitable excipients can be mixed with the active ingredient, for example, by using a wetting agent such as water or a binder syrup to mix the active ingredient and the suitable diluent, disintegrant, and binder polymer together forming small pellets and granules; sizing and drying the granules, and then adding a lubricant for mixing.

In some embodiments, the active ingredient and excipient are mixed and then the mixture is subjected to centrifugal granulation to form micropellets, Alternatively, solid dispersion can be used to prepare micropellets.

In some embodiments, the micropellets further comprise a blank pellet core. The blank pellet core can be a commercially available blank pellet core, or it can be a self-made blank pellet core. For example, the blank pellet core can be a sugar pellet or a microcrystalline cellulose pellet. During the preparation the micropellets, the active ingredient is dissolved or mixed with the excipient to form a suspension, and the suspension is then coated on the blank pellet core in a solid dispersion manner.

The solid dispersion is carried out by applying a solid dispersant. The solid dispersant is selected from the group consisting of hydroxypropyl methylcellulose, hydroxypropyl cellulose, xanthan gum, povidone K30-90, PEG series, etc., or any combination of the above. It would be appreciated other suitable solid dispersant may also be applied depending on the conditions.

Preferably, the weight ratio of the active ingredient to the excipient is about 1:0.05 to about 1:100, about 1:10 to about 1:50, or about 1:0.1 to about 1:2. Specifically, the weight ratio of the active ingredient to a carrier material is about 1:0.05 to about 1:100, and is preferably 1:10-1:50 for the drug coating micropellets, or preferably 1:0.1-1:2 for drug-containing small pellets prepared by centrifugal granulation.

In some embodiments, the enteric outer layer is an enteric coating layer coating the micropellets. The enteric coating layer comprises hypromellose phthalate (HPMCP), cellulose acetate phthalate (CAP), acrylic resin II, acrylic resin III, and methacrylic acid and methyl methacrylate copolymer, or any combination thereof. Preferably, the weight of the enteric coating layer is about 3% to about 20% of the weight of the micropellets, more preferably about 8% to about 15%.

Coating can be applied to prepare the micropellets. In a coating pan or a fluidized bed coating apparatus, a solution of the enteric coating material is sprayed onto the micropellets. Further, before coating the enteric coating layer, an isolation layer can be coated first to increase the stability of the active ingredient. The isolation layer can be selected from water-soluble coating materials such as povidone (PVP), hydroxypropyl methylcellulose (HPMC), and OPADRY series. Depending on the properties of the polymer used, the solvent can be an organic solvent or an aqueous solvent.

In some embodiments, the micropellets with the enteric coating layer are further encapsulated in a gastric-soluble capsule. In some other embodiments, the micropellets without an enteric coating layer are encapsulated in an enteric capsule. Both preparation methods can achieve a controlled-release effect, so that the pharmaceutical composition does not release or releases a minimal amount of drug (less than 20%) in the stomach, and after entering the intestine, the enteric coating begins to dissolve and gradually release the drug under appropriate pH conditions, thereby exerting a therapeutic effect.

One feature of the controlled-release pharmaceutical composition of the present invention is that the dissolution ratio of the drug during the first release period is 0-20% of the total amount of the drug. The first release process is an in vitro release rate determination in a release medium in the pH 1-4 range, where the dissolution process is from 0 to 120 minutes in the experiment, the Chinese Pharmacopoeia 2020 edition provides a suitable release rate determination experiment for determining the release rate of enteric formulations in its Appendix. The determination method is carried out in a dissolution apparatus at 37° C. in a release medium, or with modifications known to those skilled in the art. A second release process is that under the condition of an in vitro dissolution experiment with a release medium in the pH 5.8-8.0 range, the dissolution distribution continues in the time period after 120 minutes.

The present invention further provides a method for preparing the above-mentioned controlled-release pharmaceutical composition, which comprises: a) mixing the active ingredient with the excipient to form a mixture, coating a blank pellet core in a solid dispersion manner to form micropellets, or granulating to form granular micropellets;

b) coating the micropellets with an enteric material to form the enteric outer layer on an exterior of the micropellets, thereby achieving no release or a minimal release of drug (less than 20%) in the stomach, and after entering the intestine, the enteric coating begins to dissolve and gradually release the drug under appropriate pH conditions.

In some embodiments, the method further comprises, before step b), forming an isolation layer on a surface of a drug-containing core with an isolation material.

As mentioned above, the present invention include at least two preparation methods: one is to prepare the active ingredient and excipient into enteric micropellets and then encapsulate them in gastric-soluble capsules; the other is to provide uncoated granules of the active ingredient and excipient, and directly encapsulate them in enteric capsules. Both preparation methods can achieve a therapeutic effect.

It can be understood that the controlled-release pharmaceutical composition of the present invention can be used in the preparation of a medicament for treating inflammatory bowel disease, especially colitis, preferably ulcerative colitis.

Preferably, the controlled-release pharmaceutical composition of the present invention is an oral capsule formulation. Therefore, the invention provides at least three forms for administration, including micropellets with an enteric coating, enteric capsules containing micropellets, and gastric-soluble capsules containing enteric-coated micropellets. The controlled-release pharmaceutical composition does not release or releases a minimal amount of drug in the stomach, and after entering the intestine, the enteric coating begins to dissolve and gradually release the drug under appropriate pH conditions.

In a further aspect, the invention provides a method of treating a disease in a subject in need thereof. The subject is preferably a mammal and particularly a human. The subject is suffering from or at risk of suffering from an inflammatory bowel disease. The method includes a step of administering the controlled-release pharmaceutical composition of the present invention to the subject.

In an embodiment, the inflammatory bowel disease is colitis and preferably ulcerative colitis. In an embodiment, the composition is provided in an oral capsule formulation.

### Example 1

### Safety of Indigo Naturalis, Indigo, and Indirubin in Normal and Colitis Model Mice

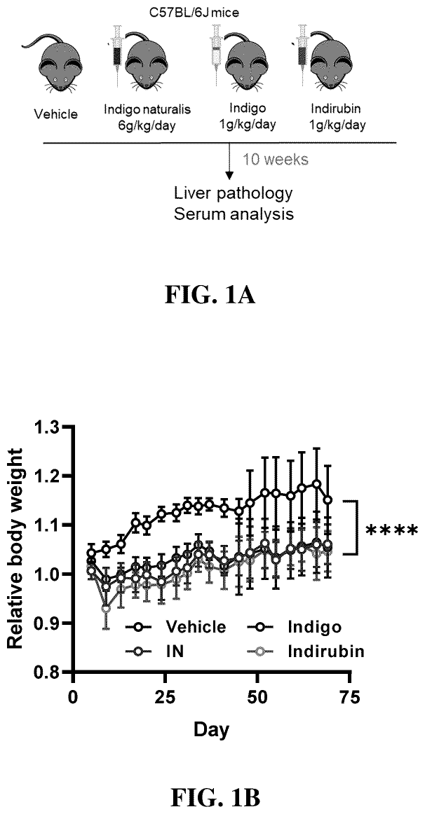

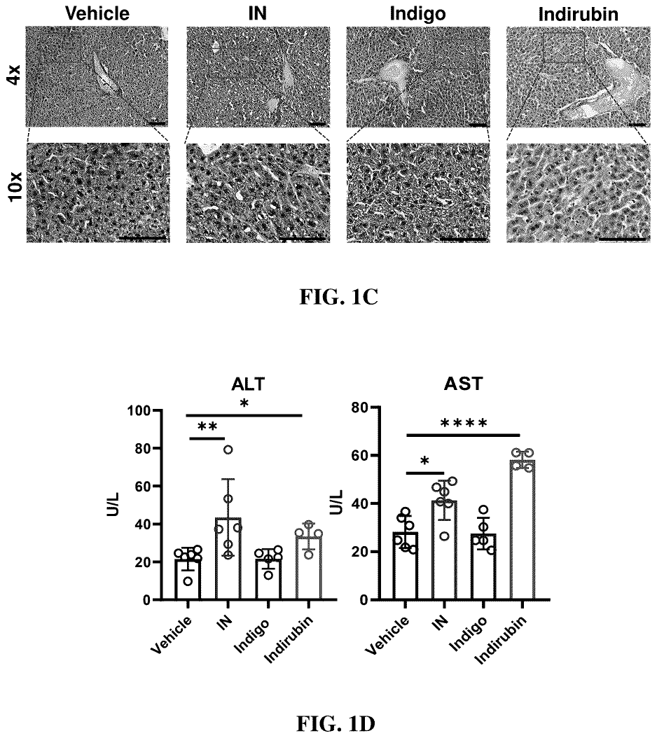

An animal model was established to simulate and determine the liver toxicity of Indigo Naturalis, indigo, and indirubin on healthy subjects. As shown in FIG. 1A, 8-week-old male C57BL/6J mice were orally administered indigo (ING) and indirubin (INB) at a dose of 1 g/kg/day, and Indigo Naturalis (IN) at a dose of 6 g/kg/day. The administered IN dose is about 10 times the human equivalent dose. The three drugs were administered orally for 10 weeks. FIG. 1B shows that the relative body weight of all treated groups of mice has no significant difference. The HE histological staining in FIG. 1C shows that no histological changes were observed in the liver in the control group and the treated groups. FIG. 1D shows that the levels of alanine aminotransferase (ALT) and aspartate aminotransferase (AST) in all treated groups showed no significant changes. These results indicate that Indigo Naturalis, indigo, and indirubin cause minimal liver damage to C57BL/6 healthy mice.

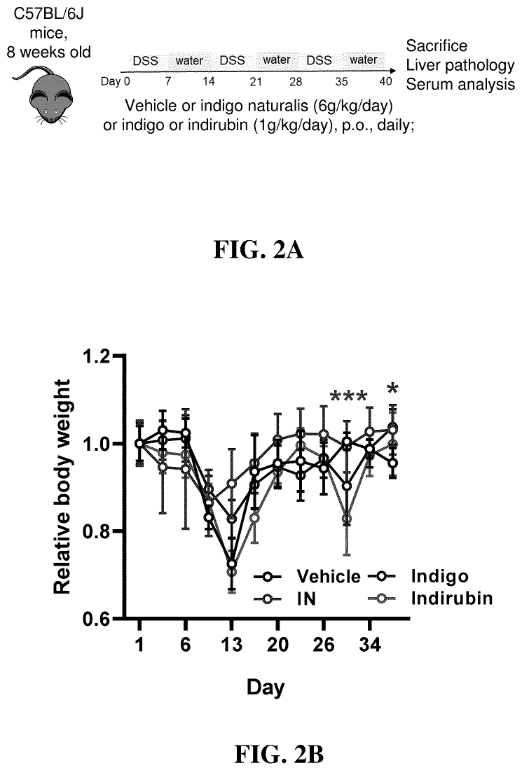

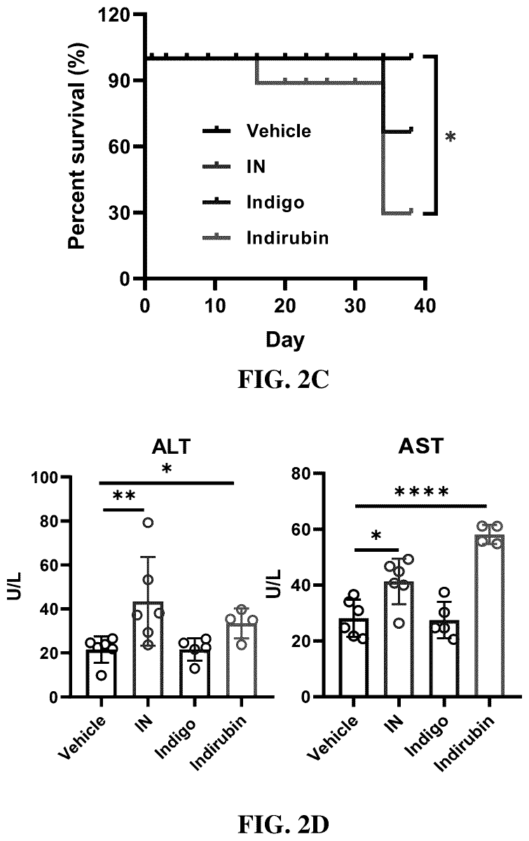

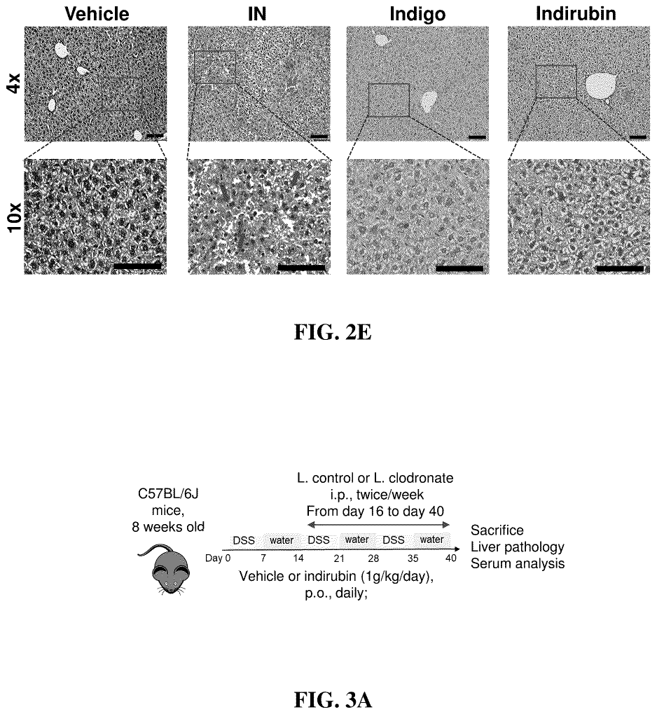

In addition, a chronic colitis animal model was established to study the effects of Indigo Naturalis, indigo, and indirubin on patients with chronic colitis, especially the effect on the liver. As shown in FIG. 2A, 1.8% dextran sulfate sodium salt (DSS) was used to induce a chronic colitis model in 8-week-old male C57BJ/6 mice. The mice were given 1.8% DSS for 7 days, followed by sterile water for 7 days as one cycle, for a total of 3 cycles. The colitis model mice were given the same high doses of IN, ING, or INB for six weeks. As shown in FIG. 2C, some of the drug-treated mice died during the experiment. FIG. 2D shows that compared with the control group mice, the mice treated with IN and INB showed significantly increased levels of ALT and AST (which are indicators of liver damages). The H&E histological analysis in FIG. 2E shows that the presence of INB caused swelling, lytic necrosis, and atrophy of mouse liver cells. These findings indicate that the drugs may increase the risk of inducing liver damage in mice with chronic colitis.

### Example 2

### Intestinal Macrophage Phagocytosis Mechanism in Colitis Mice Leads to Abnormal Drug Elevation and Liver Toxicity

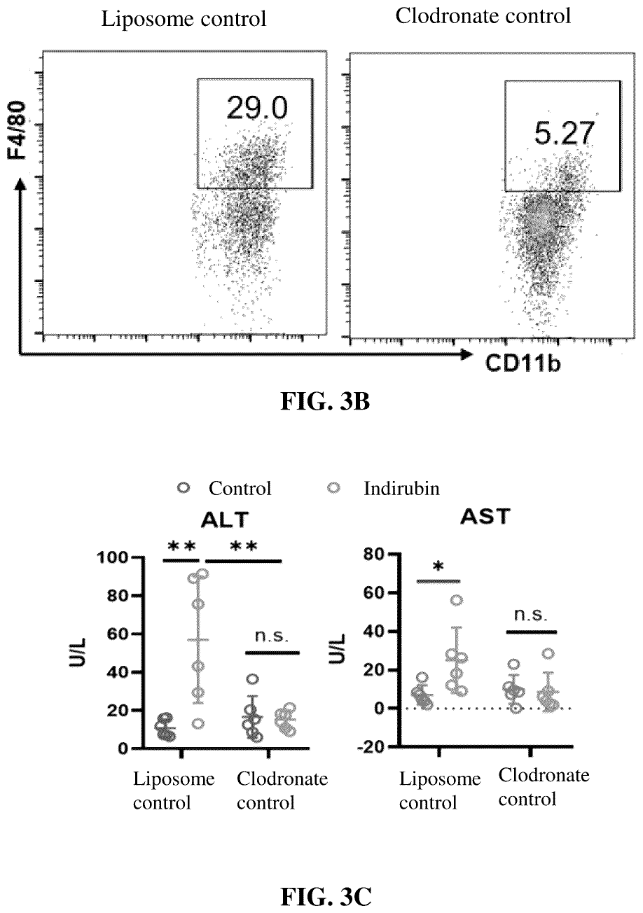

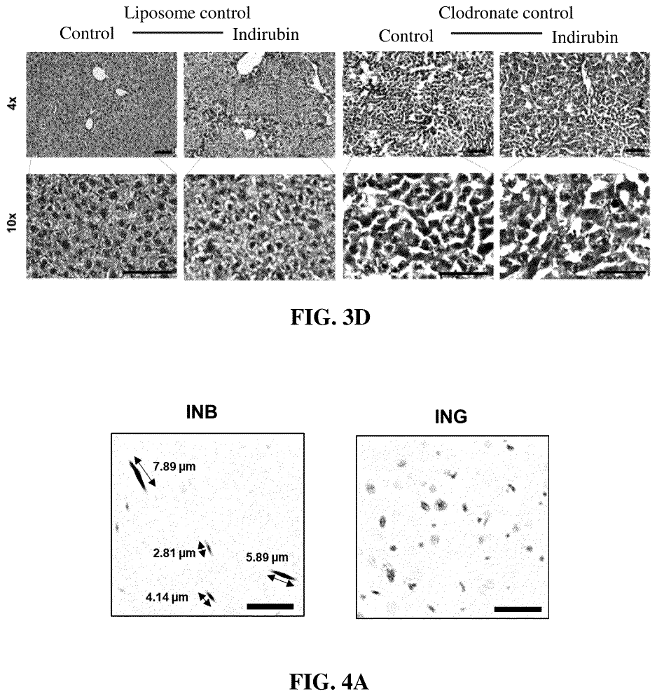

Macrophages are responsible for identifying, internalizing, and clearing dead cells or foreign particles in the body. To determine the role of macrophages in drug-induced liver injury, as shown in FIG. 3A, liposomes loaded with clodronate were used to treat and deplete phagocytic macrophages in colitis mice, and then the effect of indirubin on the animal's liver was examined. FIG. 3B shows that after 24 hours of using clodronate liposomes, macrophages were reduced by more than 90%. Using the same dose, in mice treated with control liposomes and untreated mice, it was found that after macrophage depletion, colitis mice showed significantly reduced ALT and AST elevation caused by indirubin (see FIG. 3C), and the H&E histological analysis in FIG. 3D showed no abnormal pathological changes between the control group and the treatment group. This indicates that in colitis mice, intestinal macrophages can take up the drug, leading to drug-induced liver damage.

### Examples 3-5

Preparation of Solid Dispersion Drug-Containing Granules with Micropellets of Different Particle Sizes as Carriers

A specified amount of drug is placed in a 200 ml small beaker, followed by the addition of an appropriate volume of solvent. The mixture is stirred thoroughly to obtain a uniform solution. Subsequently, the prepared solid dispersant solution is added and mixed thoroughly. After that, the resultant mixture is passed through a 100-mesh sieve to remove large particles, yielding a uniform and slightly viscous liquid for use.

A specified amount of blank small pellets is placed in a coating pan. The blower is activated to ensure gentle fluidization of the pellets within the pan. The temperature is maintained at approximately 50° C., with the pan rotation speed set to 18-20 rpm and the spray rate adjusted to 3 mL/min. Initially, a 1% HPMC solution is applied as a fixing layer to harden the blank small opellets, enhancing their mechanical strength and preventing breakage during subsequent drug and film coating steps. The prepared drug solution is then sprayed using the same coating parameters. The coating process may be adjusted in real time based on pellet flow behavior to prevent agglomeration. Upon completion of drug solution spraying, the pellets are dried in the coating pan for 20 minutes. The resulting drug-loaded micropellets are collected, treated with a small amount of talc to eliminate static electricity, sieved to remove fine powder, and weighed for content determination, yielding solid dispersion drug-containing micropellet granules.

| | Example 3 | Example 4 | Example 5 |
|---|---|---|---|
| Drug | Indigo 5.5 g | Indigo 6.0 g | Indigo 6.5 g |
| Blank pellet core | 14-16 mesh | 20-50 mesh | 40-50 mesh |
| Blank pellet core | 100 g | 150 g | 150 g |
| weight | | | |
| Binder/Dispersant | 2% HPMC | 10% Povidone | 2% HPMC |
| Solvent | (60% ethanol) | (70% ethanol) | (60% ethanol) |
| | 70 g | 50 g | 50 g |

### Examples 6-7

### Preparation of Solid Dispersion Drug-Containing Granules of Different Drugs

Following the procedures outlined in Examples 3-5, various drugs were employed to prepare solid dispersion drug-loaded micropellet granules.

| | Example 6 | Example 7 |
|---|---|---|
| Drug | Indirubin 4.5 g | Indigo Naturalis 5.5 g |
| Blank pellet core | 40-50 mesh | 20-50 mesh |
| Blank pellet core weight | 100 g | 150 g |
| Binder/Dispersant solvent | 2% HPMC (60% | 2% HPMC (60% |
| | ethanol) 50 g | ethanol) 50 g |

### Examples 8-9

### Preparation of Drug-Containing Small Pellets

First, the excipients and the raw material drug—indigo or indirubin—are passed through an 80-mesh sieve and weighed according to the prescribed amounts. The components are then thoroughly mixed and transferred into a BZJ-360 coating and granulating machine. After powering on the device, the main machine speed is set to 100 rpm, the slurry spray rate to 1.8 mL/min, the powder feed rate to 12 g/min, and the hot air temperature to 38-44° C. Granulation is carried out under these conditions until the desired particle size is achieved. The granules are dried in the coating pan for 30 minutes, then transferred to a 60° C. oven for an additional 4 hours of drying. Finally, 18-22 mesh pellet cores are sieved and collected for subsequent use.

| | Example 8 | Example 9 |
|---|---|---|
| Drug | Indirubin 100 g | Indigo 100 g |
| Starch | 40 g | 40 g |
| Lactose | 40 g | 50 g |
| Binder | 20% syrup 50 ml | 10% PVP 100 ml |

### Examples 10-17

### Preparation of Enteric Micropellets

A prescribed amount of drug-containing small pellets is placed in a coating pan, and the blower is activated to ensure the small pellets flow slowly in the pan. The temperature is set to about 50° C.-55° C., the coating pan speed is set to 20-30 rpm, and the spray speed is set to 3-5 ml/min. Initially, 25 ml of 1% HPMC solution is apply for coating as an isolation layer to enhance the mechanical strength of the drug-containing small pellets, so as to prevent their breakage during the enteric film coating process. Using the same procedure and coating parameters, apply the prepared enteric coating solution to the small pellets. The coating process may be adjusted in real time based on pellet flow to prevent sticking. After spraying, dry the small pellets in the coating pan for 10-20 minutes. Once dried, collect the small pellets, remove fine powder by sieving, weigh and determine drug content, and encapsulate as needed to produce enteric formulations of varying specifications.

| Examples | Example 10 | Example 11 | Example 12 | Example 13 |
|---|---|---|---|---|
| Drug-containing | Example 5 | Example 5 | Example 6 | Example 6 |
| micropellet source | | | | |
| Drug-containing | 50 g | 46 g | 25 g | 25 g |
| micropellet weight | | | | |
| Enteric coating | 10% Eudragit | 10% Eudragit | 10% Eudragit | 10% Eudragit |
| | L100, 140 g | S100, 138 g | L100, 60 g | S100, 50 g |
| Triethyl citrate | 0.6%, 3 g | 0.6%, 2.5 g | 0.6%, 2 g | 0.6%, 1.5 g |
| Talc | Appropriate | Appropriate | Appropriate | Appropriate |
| | amount | amount | amount | amount |
| Examples | Example 14 | Example 15 | Example 16 | Example 17 |
| Drug-containing | Example 8 | Example 9 | Example 8 | Example 9 |
| micropellet source | | | | |
| Drug-containing | 45 g | 45 g | 40 g | 40 g |
| micropellet weight | | | | |
| Enteric coating | 10% Eudragit | 10% Eudragit | 10% Eudragit | 10% Eudragit |
| | L100, 140 g | S100, 138 g | RL100, 140 g | RS100, 138 g |
| Triethyl citrate | 0.6%, 3 g | 0.6%, 2.5 g | 0.6%, 3 g | 0.6%, 2.5 g |
| Talc | Appropriate | Appropriate | Appropriate | Appropriate |
| | amount | amount | amount | amount |

### Example 18

### Preparation of Enteric Capsules

Weigh different weights of the drug-containing micropellets from the examples, and pack them into enteric capsule shells of different types and sizes to obtain enteric formulations with different specifications. Alternatively, pack the small pellets, after enteric coating, into ordinary capsules of different specifications to prepare the enteric formulations.

| Drug-containing | | | | | | |
|---|---|---|---|---|---|---|
| micropellet source | Example 3 | Example 3 | Example 4 | Example 5 | Example 8 | Example 9 |
| Capsule shell | pH 6.8 | pH 7.2 | pH 7.6 | pH 7.8 | pH 6.8 | pH 6.8 |
| dissolution pH | soluble | soluble | soluble | soluble | soluble | soluble |
| Capsule size No. | 00# | 0# | 1# | 2# | 2# | 0# |
| Fill weight | 800 mg | 500 mg | 300 mg | 200 mg | 200 mg | 500 mg |
| Formulation | 50 mg | 30 mg | 20 mg | 5 mg | 5 mg | 200 mg |
| specification | | | | | | |

### Example 19

### Determination of the Release Rate of Drug Micropellet Samples of Different Particle Sizes

Pellet samples of various mesh sizes were tested using three cups per sample, with two capsules in each cup. Dissolution and release rate were determined in accordance with the Chinese Pharmacopoeia (2020 Edition, Part Four, General Rule 0931, First Method, Basket Method), using 500 mL of buffer solution at different pH levels as the dissolution medium and a rotation speed of 75 rpm. Samples were collected at 10, 20, 30, 45, 60, 100, and 120 minutes, filtered, and the resulting filtrate was used as the test solution. Separately, approximately 20 mg of reference standard was accurately weighed and placed into a 50 mL volumetric flask. An appropriate volume of the corresponding buffer solution was added, and the mixture was sonicated for 10 minutes to dissolve the standard. After cooling to room temperature, the solution was diluted to volume with the same buffer and mixed thoroughly. A 1 mL aliquot was then transferred to a 10 mL volumetric flask, diluted to volume with dissolution medium, and mixed well to prepare the reference solution. Absorbance of both the test and reference solutions was measured at dual wavelengths, and the dissolution amount was calculated. The results demonstrated that both the capsule and coating formulations achieved the intended enteric dissolution performance

| Source | Example 3 | Example 5 | Example 10 | Example 11 | Example 12 | Example 13 | Example 17 |
|---|---|---|---|---|---|---|---|
| Enteric | 20 mg | 20 mg | 20 mg | 20 mg | 20 mg | 20 mg | 100 mg |
| formulation | — | pH 6.8 | Eudragit | Eudragit | Eudragit | Eudragit | Eudragit |
| | | dissolving | L100 | S100 | L100 | S100 | RS100 |
| | | capsule | | | | | |
| 1 hour gastric | >90% | <15% | <15% | <15% | <15% | <15% | <15% |
| fluid | | | | | | | |
| dissolution | | | | | | | |
| Intestinal fluid | — | pH 6.8 | pH 6.8 | pH 7.6 | pH 6.8 | pH 7.6 | pH 6.8 |
| medium | | | | | | | |
| Intestinal fluid | — | 33.2% | 55.5% | 58.7% | 98.7% | 89.9% | 45.6% |
| 20 minutes | | | | | | | |
| Intestinal fluid | — | 70.6% | 65.7% | 64.3 | — | — | 75.8% |
| 120 minutes | | | | | | | |

### Example 20

### Phagocytosis Results of Macrophages on Different Drug Forms

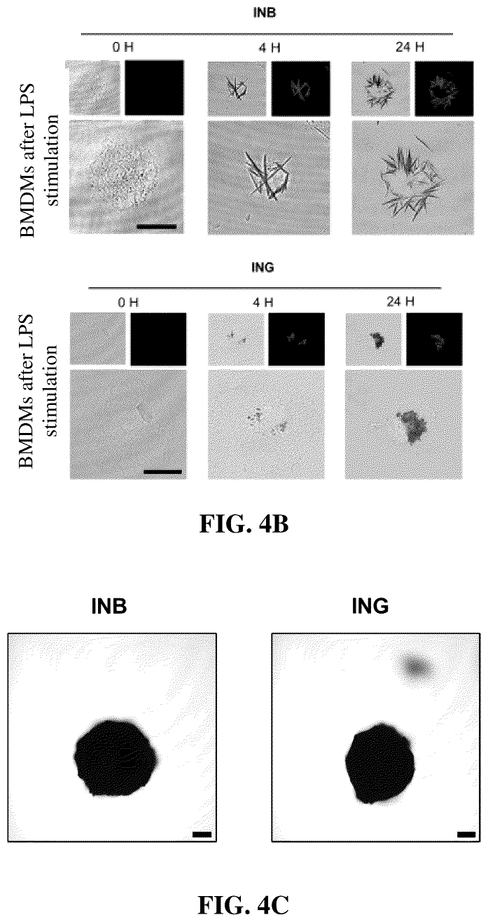

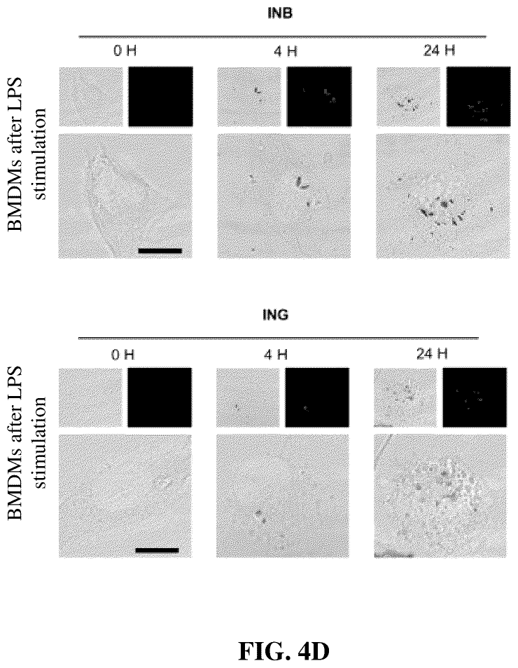

Phagocytosis is a unique function of macrophages and is highly dependent on the size and shape of particles. As shown in FIG. 4A, the inventors found that indirubin will form needle-shaped particles with a size from 3 to 8 μm in the aqueous phase, and tends to aggregate after long-term culture. Unlike indirubin, indigo always maintains a granular shape. FIG. 4B shows that after 4 hours of incubation with indirubin and indigo, bone marrow-derived macrophages (BMDM) can internalize indirubin and indigo, and further microscopic observation confirms that indirubin phagocytosis increases proportionally with time. Afterwards, comparing the appearance of the enteric-coated micropellets of Example 11 at the same drug amount (50μ g/well) (see FIG. 4C) in a macrophage phagocytosis experiment, the experimental results in FIG. 4D show that the pharmaceutical composition of the present invention can reduce the phagocytosis phenomenon of macrophages.

### Example 21

### In Vivo Pharmacodynamics and Safety of Different Drug Forms in Mice

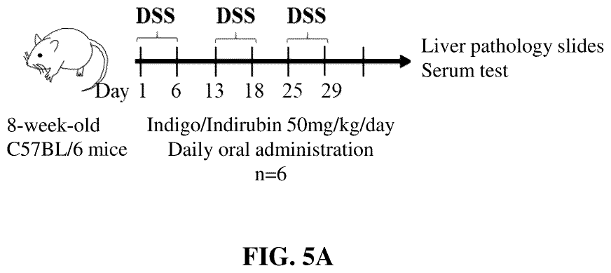

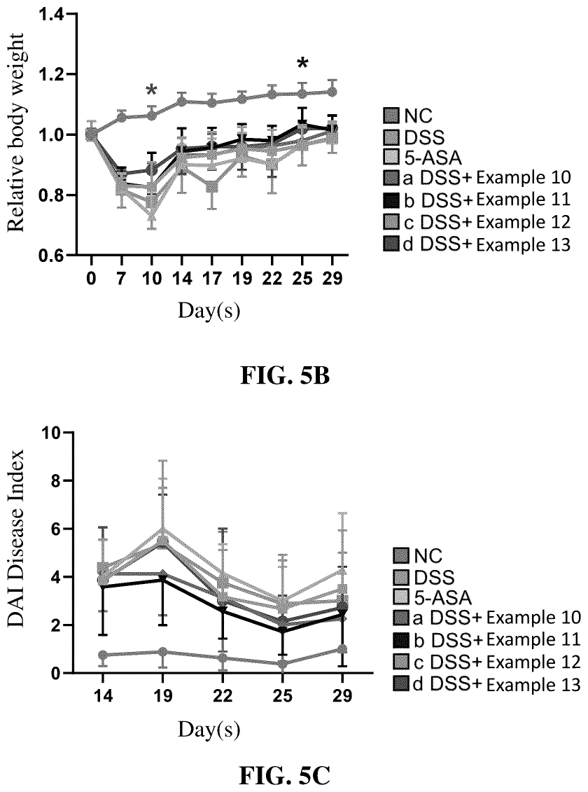

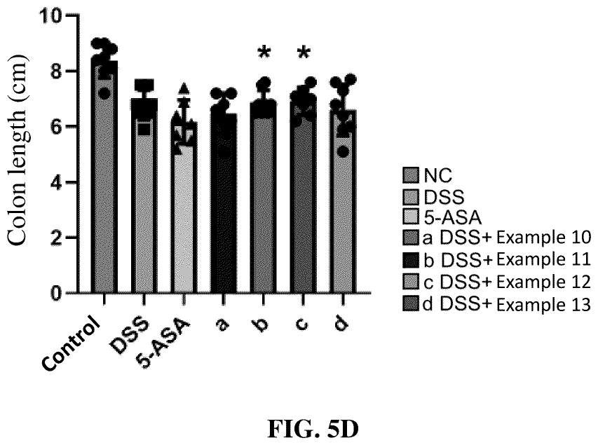

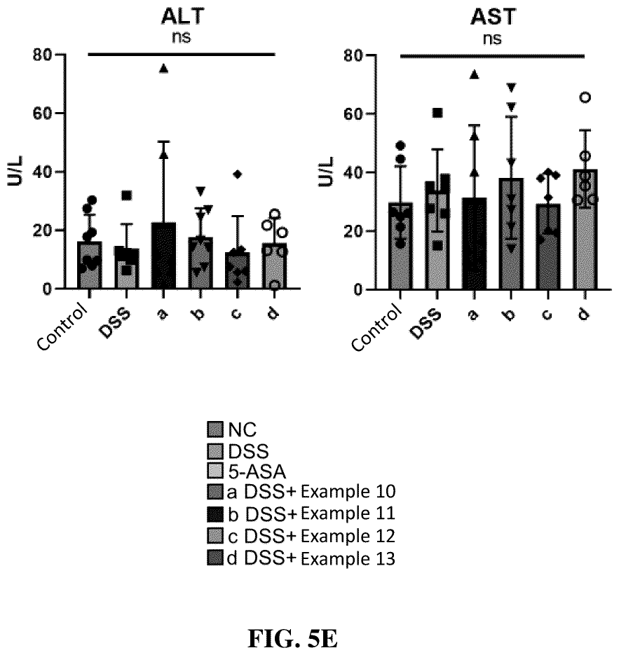

As shown in FIG. 5A, a 3-cycle experimental protocol was employed. Mice were administered a 1.8% DSS solution for 5 days, followed by sterile water for 7 days to complete one cycle, repeated for a total of three cycles. Throughout the experiment, body weight, fecal occult blood volume, and fecal water content were monitored to assess the disease activity index (DAI). As illustrated in FIGS. 5B-5E, treatment with indigo and indirubin micropellets significantly mitigated DSS-induced weight loss and disease severity. Post-sacrifice, colon length measurements revealed that the micropellet treatment notably increased colon length. Additionally, serum analysis of ALT and AST levels showed no significant differences between the indigo and indirubin micropellet groups and either the control or model groups.

In summary, the inventor evaluated the toxicity of Indigo Naturalis, indigo, and indirubin administered by gavage to both normal and colitis model mice. The results demonstrated significant toxicity following oral administration in colitis mice, particularly hepatotoxicity, as evidenced by elevated AST and ALT levels. Further comparison under conditions of intestinal macrophage presence and depletion confirmed that macrophage-mediated phagocytosis is the primary mechanism underlying abnormal drug accumulation and liver toxicity. Based on these findings, macrophages exhibit the highest phagocytic activity toward particles ranging from 0.3 to 10 μm. To address this issue, the present invention proposes a strategy for treating colitis with Indigo Naturalis or its main active component-indigo and indirubin—by minimizing or avoiding intestinal macrophage phagocytosis. This is achieved through a solid dispersion technique that coats the drug onto micropellets or directly formulates micropellet cores, followed by enteric coating or encapsulation to enable pH-controlled release. The approach reduces macrophage uptake through three mechanisms: (1) enteric formulations with high pH-triggered release limit drug exposure in the upper intestine and target release to the lower intestine, thereby shortening overall intestinal exposure time; (2) coating the drug onto blank small pellets or combining it with excipient to form drug-containing pellets renders the particles less susceptible to phagocytosis; and (3) the solid dispersion of drug-containing pellets reduces local crystallization, further decreasing macrophage uptake.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A controlled-release pharmaceutical composition containing Indigo Naturalis or its main active ingredient, the composition comprising micropellets; wherein the micropellets comprise an active ingredient and one or more pharmaceutically acceptable excipients, the active ingredient comprises Indigo Naturalis, indigo, indirubin, or any combination thereof; and the micropellets have an enteric outer layer.

2. The controlled-release pharmaceutical composition according to claim 1, wherein the micropellets further comprise a blank pellet core, and the active ingredient and the excipient are mixed and coated on the blank pellet core in a solid dispersion manner.

3. The controlled-release pharmaceutical composition according to claim 1, wherein the micropellets are granules formed by mixing the active ingredient and the excipient, and granulation.

4. The controlled-release pharmaceutical composition according to claim 1, wherein a weight ratio of the active ingredient to the excipient is about 1:0.05 to about 1:100, about 1:10 to about 1:50, or about 1:0.1 to about 1:2.

5. The controlled-release pharmaceutical composition according to claim 1, wherein the enteric outer layer is an enteric coating layer coating the micropellets; the enteric coating layer comprises a material selected from hypromellose phthalate (HPMCP), cellulose acetate phthalate (CAP), acrylic resin II, acrylic resin III, and methacrylic acid and methyl methacrylate copolymer, or any combination thereof.

6. The controlled-release pharmaceutical composition according to claim 5, wherein a weight of the enteric coating layer is 3% to 20% of a weight of the micropellets; micropellets and the enteric coating layer; micropellets with the enteric coating layer are further encapsulated in a gastric-soluble capsule.

7. The controlled-release pharmaceutical composition according to claim 1, wherein the enteric outer layer is an enteric capsule for encapsulating the micropellets.

8. A method for preparing the controlled-release pharmaceutical composition according to claim 1, comprising:
a) mixing the active ingredient with the excipient to form a mixture, coating a blank pellet core in a solid dispersion manner to form micropellets, or granulating to form granular micropellets;
b) coating the micropellets with an enteric material to form the enteric outer layer on an exterior of the micropellets.

9. The method according to claim 8, further comprising, before step b), forming an isolation layer on a surface of a drug-containing core with an isolation material.

10. A method of treating an inflammatory bowel disease, comprising administering the controlled-release pharmaceutical composition according to claim 1 to a subject in need thereof.

11. The method according to claim 10, wherein the inflammatory bowel disease is ulcerative colitis.

12. The method according to claim 10, wherein the composition is provided in an oral capsule formulation.
